(19) United States
(12) Patent Application Publication
(10) Pub. No.: US 2019/0295698 A1
(43) Pub. Date: Sep. 26, 2019

(54) PATIENT CARE INTEGRATION SYSTEM AND METHODS

(71) Applicant: Unite USA Inc. dba Unite Us, New York City, NY (US)

(72) Inventor: Eshed Doni, Tenafly, NJ (US)

(73) Assignee: Unite USA Inc. dba Unite Us, New York City, NY (US)

(21) Appl. No.: 16/360,577

(22) Filed: Mar. 21, 2019

Related U.S. Application Data
(60) Provisional application No. 62/647,168, filed on Mar. 23, 2018.

(51) Int. Cl. G16H 10/60; G16H 15/00

(52) U.S. Cl. CPC G16H 10/60; G16H 80/00; G16H 15/00

(57) ABSTRACT

A patient care integration system and methods are provided which may include one or more steps, such as: receiving a request for social data of a patient via an electronic medical records graphical user interface (EMR graphical user interface) of an electronic medical records system (EMR system), the EMR graphical user interface displayed on a display screen of a client device; querying a non-medical care coordination system (NMCC system) for social data of the patient; returning social data for the patient via from the NMCC system; generating a non-medical care coordination graphical user interface (NMCC graphical user interface) within the EMR graphical user interface, the NMCC graphical user interface displaying the social data on the display screen of the client device; and creating a social referral for the patient via the NMCC graphical user interface in the NMCC system.

### CROSS REFERENCE TO RELATED APPLICATIONS

This application claims priority to and the benefit of the filing date of U.S. Provisional Application No. 62/647,168, filed on Mar. 23, 2018, entitled “METHOD AND SYSTEM FOR INTEGRATING NON-MEDICAL CARE COORDINATION SYSTEM WITH ELECTRONIC MEDICAL RECORD SYSTEMS”, which is hereby incorporated by reference in its entirety.

### FIELD OF THE INVENTION

This patent specification generally relates to the field of systems and methods configured for integrating two or more patient care information systems. More specifically, this patent application relates to integrating non-medical care coordination systems within electronic medical records systems in a way that combines medical and non-medical patient information.

### BACKGROUND

Research has proven that the social determinants of health are the predominant factor in affecting healthcare outcomes.

These findings fuel the efforts to identify the patient's social needs in parallel to addressing their medical needs, in order to optimize the outcomes of the overall care, medical and non-medical, the patient receives.

Various initiatives tie together social and medical needs through identifying the social needs of medical patients aiming at addressing social determinants as a means to reduce hospital readmission rates.

One of the main challenges in establishing such a process has to do with the fact that it brings together a number of professionals that work in separate distinct settings and use different systems. These professionals work at hospitals and other medical facilities, welfare agencies, city and state-run organizations, independent service providers, Nonprofits, and many more. Hospital personnel use electronic medical records systems while the other involved parties often lack access to these systems, and vice versa, and even if hospital personnel had access to social care coordination systems, they would be reluctant to use yet another system as they are overloaded already and using an additional system requires training and has a learning curve.

Therefore, a need exists for novel computer-implemented systems and methods for providing an easy-to-use integrated solution that combines medical and social needs while various stake holders continue to use different systems.

### BRIEF SUMMARY OF THE INVENTION

A patient care integration system and methods are provided that facilitate care coordination and that combine medical and non-medical care. The system and method may enable integrating non-medical care coordination systems within electronic medical records systems in a seamless manner that provides superior user experience to users of both systems while providing a coherent and comprehensive picture that combines medical and non-medical patient information. Additionally, the system and methods may allow the initiation of non-medical care episodes by medical professionals. This integration also enables the tracking of non-medical care episodes through an electronic medical records system.

According to one embodiment consistent with the principles of the invention, a computer implemented method for accessing social data of a patient is provided. In some embodiments, the method may include the steps of: an electronic medical records system (EMR system) sending a request to a non-medical care coordination system (NMCC system) to present social data of a patient via the electronic medical records graphical user interface (EMR graphical user interface) of that EMR system, and the EMR graphical user interface may be displayed on a display screen of a client device; the NMCC system querying for social data of the patient; returning social data for the patient via from the NMCC system; and generating a non-medical care coordination graphical user interface (NMCC graphical user interface) within the EMR graphical user interface, the NMCC graphical user interface displaying the social data on the display screen of the client device.

According to another embodiment consistent with the principles of the invention, a computer implemented method for modifying social data of a patient is provided. In some embodiments, the method may include: generating a non-medical care coordination graphical user interface (NMCC graphical user interface) of a non-medical care coordination system (NMCC system) within an electronic medical records graphical user interface (EMR graphical user interface) of an electronic medical records system (EMR system) to provide social data of a patient to a user via a display screen of a client device; and creating a social referral for the patient via the NMCC graphical user interface in the NMCC system.

According to yet another embodiment consistent with the principles of the invention, a computer implemented patient care integration method is provided. In some embodiments, the method may include the steps of: an electronic medical records system (EMR system) sending a request to a non-medical care coordination system (NMCC system) to present social data of a patient via the electronic medical records graphical user interface (EMR graphical user interface) of that EMR system, and the EMR graphical user interface may be displayed on a display screen of a client device; the NMCC system querying for social data of the patient; returning social data for the patient via the NMCC system; generating a non-medical care coordination graphical user interface (NMCC graphical user interface) within the EMR graphical user interface, the NMCC graphical user interface displaying the social data on the display screen of the client device, and creating a social referral for the patient via the NMCC graphical user interface in the NMCC system.

In still further embodiments, a computer implemented patient care integration method may include the step of displaying a screening questionnaire for the patient to the user via the display screen of a client device. The method may further include the step of receiving screening data for the patient, and also may include the step of identifying a social need for the patient from the screening data via an interface engine.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts an illustrative example of some of the components and computer implemented methods which may be found in a patient care integration system according to various embodiments described herein.

FIG. 2 illustrates a block diagram showing an example of a server which may be used by the system as described in various embodiments herein.

FIG. 3 shows a block diagram illustrating an example of a client device which may be used by the system as described in various embodiments herein.

FIG. 4 depicts a block diagram illustrating some engines and components of a patient care integration system according to various embodiments described herein.

FIG. 5 illustrates an example screenshot of an electronic medical records graphical user interface according to various embodiments described herein.

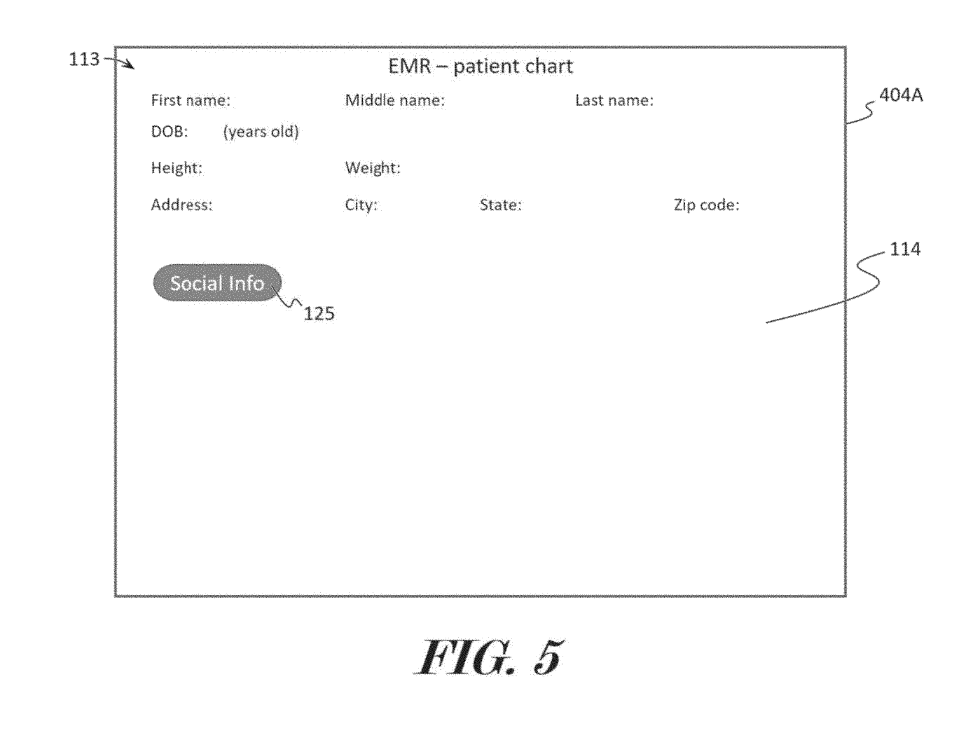

FIG. 6 shows an example screenshot of a non-medical care coordination graphical user interface embedded within a social data frame of an electronic medical records graphical user interface according to various embodiments described herein.

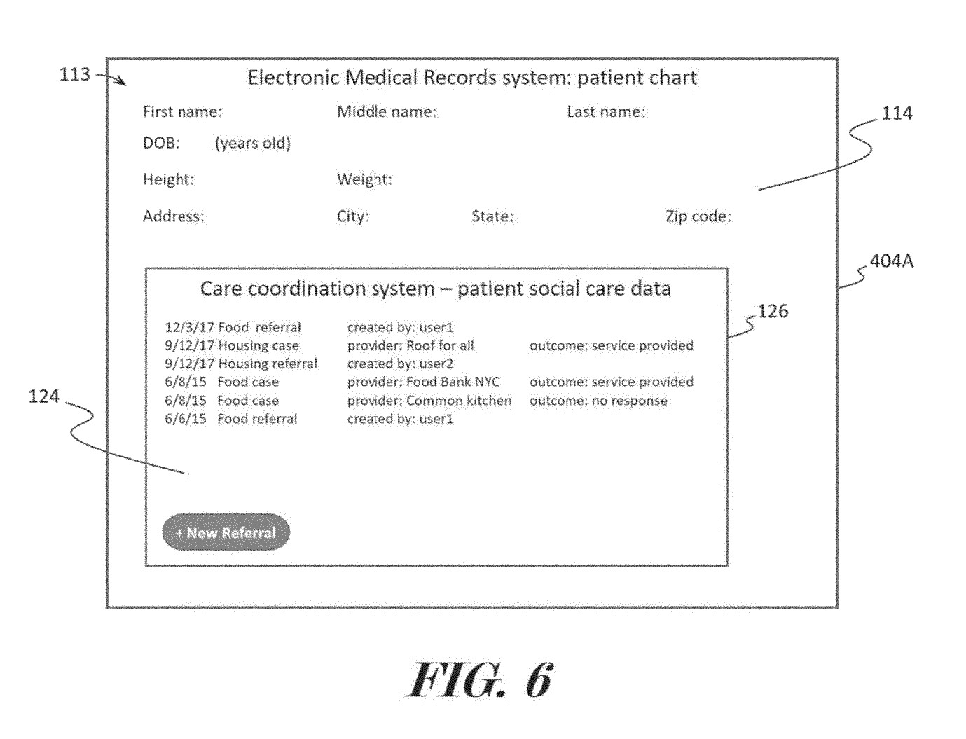

FIG. 7 depicts a block diagram of an example of a computer-implemented method of accessing social data of a patient according to various embodiments described herein.

FIG. 8 illustrates a block diagram of an example of a computer-implemented modifying social data of a patient according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

### Definitions

As used herein, the term “computer” refers to a machine, apparatus, or device that is capable of accepting and performing logic operations from software code. The term “application”, “software”, “software code”, “source code”, “script”, or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “electronic device” as used herein is a type of computer comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, servers, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anota digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VoIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New computer-implemented systems and methods configured for integrating two or more patient care information systems are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

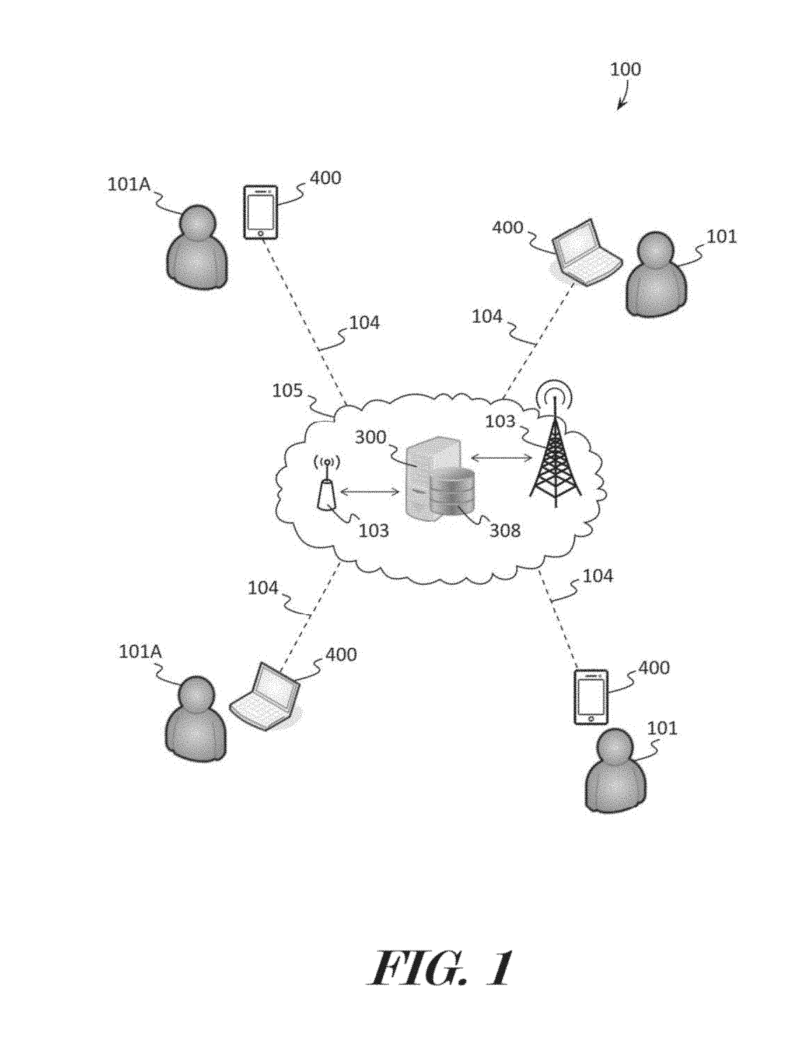

The present invention will now be described by example and through referencing the appended figures representing preferred and alternative embodiments. As perhaps best shown by FIG. 1, an illustrative example of some of the physical components which may comprise a patient care integration system (“the system”) 100 according to some embodiments is presented. The system 100 is configured to facilitate the transfer of data and information between one or more access points 103, client devices 400, and servers 300 over a data network 105. Each client device 400 may send data to and receive data from the data network 105 through a network connection 104 with an access point 103. A data store 308 accessible by the server 300 may contain one or more databases. The data may comprise any information which may describe a patient or care recipient of electronic medical records systems 110 and non-medical care coordination systems 120.

In this example, the system 100 comprises at least one client device 400 (but preferably more than two client devices 400) configured to be operated by one or more users 101. Client devices 400 can be mobile devices, such as laptops, tablet computers, personal digital assistants, smart phones, and the like, that are equipped with a wireless network interface capable of sending data to one or more servers 300 with access to one or more data stores 308 over a network 105 such as a wireless local area network (WLAN). Additionally, client devices 400 can be fixed devices, such as desktops, workstations, and the like, that are equipped with a wireless or wired network interface capable of sending data to one or more servers 300 with access to one or more data stores 308 over a wireless or wired local area network 105. The present invention may be implemented on at least one client device 400 and/or server 300 programmed to perform one or more of the steps described herein. In some embodiments, more than one client device 400 and/or server 300 may be used, with each being programmed to carry out one or more steps of a method or process described herein.

In some embodiments, the system 100 may be configured to facilitate the communication of information to and from one or more users 101, through their respective client devices 400, and servers 300 of the system 100. Users 101 of the system 100 may include medical professionals 101A, such as a healthcare provider or a non-healthcare care provider, or otherwise be associated with patient care, such as secretary or other administrative personnel.

In preferred embodiments, the system 100 may be configured to enable care coordination that integrates medical care and non-medical care to allow the initiation of non-medical care episodes by medical professionals 101A for one or more patients. This integration also enables the tracking of non-medical care episodes through an electronic medical records system (EMR system) 110. The system 100 may enable patient information to be exchanged between one or more electronic medical records systems 110 and one or more non-medical care coordination systems 120 to eliminate the need for individuals, such as medical professionals 101A, to re-enter patient information into electronic medical records systems 110 and/or non-medical care coordination systems 120.

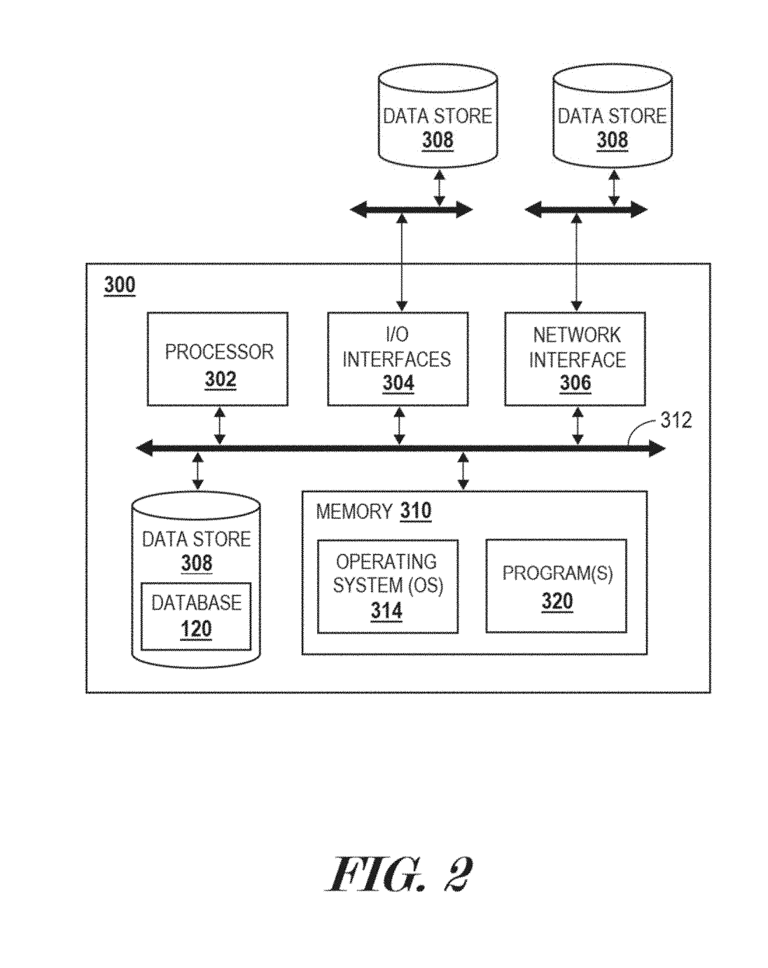

Referring now to FIG. 2, in an exemplary embodiment, a block diagram illustrates a server 300 of which one or more may be used in the system 100 or standalone and which may be a type of computing platform. The server 300 may be a digital computer that, in terms of hardware architecture, generally includes a processor 302, input/output (I/O) interfaces 304, a network interface 306, a data store 308, and memory 310. It should be appreciated by those of ordinary skill in the art that FIG. 2 depicts the server 300 in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 may be, for example but not limited to, one or more buses or other wired or wireless connections, as is known in the art. The local interface 312 may have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 may be any custom made or commercially available processor, a central processing unit (CPU), an auxiliary processor among several processors associated with the server 300, a semiconductor-based microprocessor (in the form of a microchip or chip set), or generally any device for executing software instructions. When the server 300 is in operation, the processor 302 is configured to execute software stored within the memory 310, to communicate data to and from the memory 310, and to generally control operations of the server 300 pursuant to the software instructions. The I/O interfaces 304 may be used to receive user input from and/or for providing system output to one or more devices or components. User input may be provided via, for example, a keyboard, touch pad, and/or a mouse. System output may be provided via a display device and a printer (not shown). I/O interfaces 304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 306 may be used to enable the server 300 to communicate on a network, such as the Internet, the data network 105, the enterprise, and the like, etc. The network interface 306 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 306 may include address, control, and/or data connections to enable appropriate communications on the network. A data store 308 may be used to store data.

The data store 308 is a type of memory and may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media. In one example, the data store 308 may be located internal to the server 300 such as, for example, an internal hard drive connected to the local interface 312 in the server 300. Additionally, in another embodiment, the data store 308 may be located external to the server 300 such as, for example, an external hard drive connected to the I/O interfaces 304 (e.g., SCSI or USB connection). In a further embodiment, the data store 308 may be connected to the server 300 through a network, such as, for example, a network attached file server.

The memory 310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 may include one or more software programs, each of which includes an ordered listing of executable instructions for implementing logical functions. The software in the memory 310 may include a suitable operating system (O/S) 314 and one or more programs 320.

The operating system 314 essentially controls the execution of other computer programs, such as the one or more programs 320, and provides scheduling, input-output control, file and data management, memory management, and communication control and related services. The operating system 314 may be, for example Windows NT, Windows 2000, Windows XP, Windows Vista, Windows 7, Windows 8, Windows 10, Windows Server 2003/2008/2012/2016 (all available from Microsoft, Corp. of Redmond, Wash.), Solaris (available from Sun Microsystems, Inc. of Palo Alto, Calif.), LINUX (or another UNIX variant) (available from Red Hat of Raleigh, N.C. and various other vendors), Android and variants thereof (available from Google, Inc. of Mountain View, Calif.), Apple OS X and variants thereof (available from Apple, Inc. of Cupertino, Calif.), or the like. The one or more programs 320 may be configured to implement the various processes, algorithms, methods, techniques, etc. described herein.

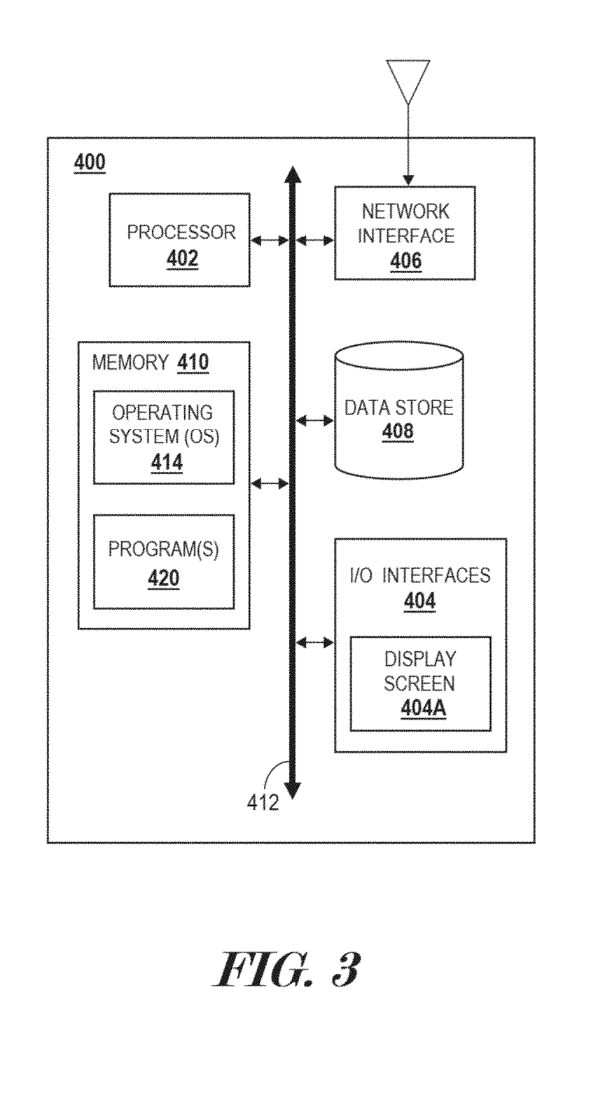

Referring to FIG. 3, in an exemplary embodiment, a block diagram illustrates a client device 400 of which one or more may be used in the system 100 or the like and which may be a type of computing platform. The client device 400 can be a digital device that, in terms of hardware architecture, generally includes a processor 402, input/output (I/O) interfaces 404, a network interface 406, a data store 408, and memory 410. It should be appreciated by those of ordinary skill in the art that FIG. 3 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

The I/O interfaces 404 can be used to receive data and user input and/or for providing system output. User input can be provided via a plurality of I/O interfaces 404, such as a keypad, a touch screen, a camera, a microphone, a scroll ball, a scroll bar, buttons, bar code scanner, voice recognition, eye gesture, and the like. System output can be provided via a display screen 404A such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker or other sound emitting device configured to emit audio notifications, a vibrational device configured to vibrate, shake, or produce any other series of rapid and repeated movements to produce haptic notifications, and/or a light emitting diode (LED) or other light emitting element which may be configured to illuminate to provide a visual notification.

The network interface 406 enables wired and/or wireless communication to an external access device or network. Any number of suitable wireless data communication protocols, techniques, or methodologies can be supported by the network interface 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions. In the example of FIG. 3, the software in the memory system 410 includes a suitable operating system (O/S) 414 and programs 420.

The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, Microsoft Windows 10, iOS (available from Apple, Inc.), webOS (available from Hewlett Packard), Blackberry OS (Available from Research in Motion), and the like.

The programs 420 may include various applications, add-ons, etc. configured to provide end user functionality with the client device 400. For example, exemplary programs 420 may include, but not limited to, electronic medical records system record keeping software, non-medical care coordination system record keeping software, a web browser, social networking applications, streaming media applications, games, mapping and location applications, electronic mail applications, financial applications, and the like. In a typical example, the end user typically uses one or more of the programs 420 along with a network 105 to manipulate information of the system 100.

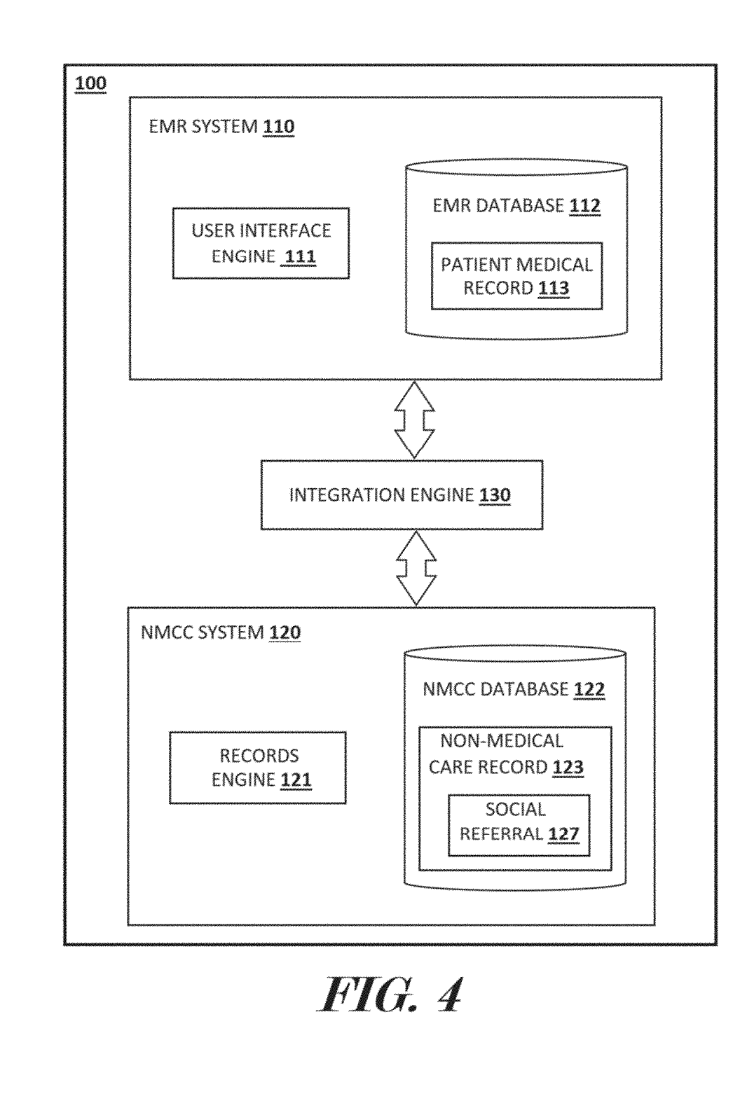

Referring now to FIG. 4 a block diagram showing some software rules engines and other components which may be found in a system 100 (FIG. 1) and which may optionally be configured to run on one or more servers 300 and/or client devices 400 that may be in communication according to various embodiments described herein are illustrated. A server 300 and client device 400 may be in wired and/or wireless electronic communication through a network 105 with a data store 308 comprising one or more databases. The engines 111, 121, 130, may read, write, or otherwise access data in one or more databases 112, 122, of one or more data stores 308. Additionally, the engines 111, 121, 130, may be in electronic communication so that data may be readily exchanged between the engines 111, 121, 130.

In this and some embodiments, one or more servers 300 and/or client devices 400 may be configured to run one or more software rules engines 111, 121, 130, of the system 100. It should be understood that the functions attributed to the engines 111, 121, 130, described herein are exemplary in nature, and that in alternative embodiments, any function attributed to any engine 111, 121, 130, may be performed by one or more other engines 111, 121, 130, or any other suitable processor logic. Similarly, data from one database 112, 122, may be stored in another database 112, 122, and/or one or more of the databases 112, 122, may be combined into a single database.

The system 100 may comprise and/or be in communication with one or more electronic medical records systems (EMR system) 110. An EMR system 110 may generally comprise a system for creating, managing, storing, and retrieving medical records for one or more patients. Typically, data of an EMR system 110 may be entered and accessed by one or more medical professionals 101A, such as doctors, nurses, physician assistants, other healthcare personnel and the like. Examples of EMR systems 110 may include Epic, Cerner, AllScripts, eClinicalWorks, Athena Health, etc.

The EMR system 110 may comprise or be in communication with one or more electronic medical records databases (EMR database) 112. A database 112 may contain one or more patient medical records 113, and each patient medical record 113 may contain personal healthcare information and other healthcare information for a particular patient. For example, a patient medical record 113 may include information such as: home and mailing address; other contact information; date of birth; weight; height; blood type; genetic information; medical conditions; medical treatments; prescriptions; diagnostic test data; and/or any other data of a patient which may be associated or used to provide medical care to a patient.

The EMR system 110 may comprise or be in communication with a user interface engine 111. In some embodiments, the user interface engine 111 may comprise or function as interface logic stored in a memory 310, 410, which may be executable by the processor 302, 402, of a server 300 and/or client device 400. Data of the EMR system 110 may be created, edited, added, or otherwise modified by a user interface engine 111. Generally, a user interface engine 111 may be configured to provide an electronic medical records graphical user interface (EMR graphical user interface) 114, such as which may be displayed on a display screen 404A of a client device 400, which a user 101 may interact with in order to create, edit, add, or otherwise modify data of the system 100, such as one or more patient medical records 113. In further embodiments, a user interface engine 111 of an EMR system 110 may send a request to a NMCC system 120, such as to a records engine 121, optionally by launching the NMCC system 120, to present social data of a patient via the EMR graphical user interface 114 of that EMR system 110, and the request may include the ID of the patient. The NMCC system 120 may then look up the social data of the patient in an EMR database 112 using the patient ID and the social data may be displayed on the EMR graphical user interface 114 optionally via the records engine 121 of the NMCC system 120 and/or optionally via the user interface engine 111 of the EMR system 110.

The system 100 may comprise and/or be in communication with one or more non-medical care coordination system (NMCC system) 120. A NMCC system 120 may generally comprise a system for creating, managing, storing, and retrieving non-medical records for one or more individuals which may be patients having a patient medical record 113. Typically, data of a NMCC system 120 may be entered and accessed by one or more non-medical professionals, such as social workers, case workers, interventionists, mental wellbeing professionals, physical wellbeing professionals, and the like and/or medical professionals 101A. Examples of NMCC systems 120 may include Unite Us, etc.

The system NMCC 120 may comprise or be in communication with one or more non-medical care coordination databases (NMCC database) 122. A database 122 may contain one or more non-medical care records 123, and each non-medical care record 123 may contain personal non-healthcare information for a particular individual that may also be a patient. For example, a non-medical care record 123 may include information such as: home and mailing address; other contact information; date of birth; social data; and/or any other data of an individual which may be associated with or used to provide non-medical care to an individual. Social data of an individual may generally be data that describes one or more social conditions, social needs, and social care episodes of that individual. This social data may further include one or more social assessments, social referrals 127, social cases, social intakes, social assistant requests, etc. A social need is a social condition that the individual has. Generally, a social need may be likened to being the social equivalent of a medical diagnosis. Common examples for social needs include housing, food, substance use, assistance with utility bills, legal advisory, etc. A social referral 127 may be a referral sent for a specific individual to a specific provider of social services. The social referral 127 may typically include demographic data about the individual, contact information for the individual, a description of the individual's social need, optionally one or more assessments that describe the individual's condition. A social referral 127 may also include one or more documents that describe the individual. An example of a social referral 127 may be a referral that is sent to MyFoodPantry for patient John Doe who was born on Jan. 1, 1989 and lives at 65 N. Moore St., New York City, N.Y. 10013, and the referral is for Emergency Food that will suffice for 14 days.

The NMCC system 120 may comprise or be in communication with a records engine 121. In some embodiments, the records engine 121 may comprise or function as records logic stored in a memory 310, 410, which may be executable by the processor 302, 402, of a server 300 and/or client device 400. Data of the NMCC system 120 may be created, edited, added, or otherwise modified by a records engine 121. Generally, a records engine 121 may be configured to receive, edit, and provide data from a NMCC database 122, such as one or more non-medical care records 123 to a user interface engine 111 via an integration engine 130. In some embodiments, a records engine 121 may be configured to provide a non-medical care coordination graphical user interface (NMCC graphical user interface) 124, such as which may be displayed on a display screen 404A of a client device 400, which a user 101 may interact with in order to create, edit, add, or otherwise modify data of the NMCC system 120, such as the social data contained in one or more non-medical care records 123. In preferred embodiments, an integration engine 130 and/or a records engine 121 may generate a NMCC graphical user interface 124 within a social data frame 126 of an EMR graphical user interface 114 so that the NMCC graphical user interface 124 may be embedded within the EMR graphical user interface 114.

The system 100 may comprise an integration engine 130 which may comprise or function as integration logic stored in a memory 310, 410, which may be executable by the processor 302, 402, of a server 300 and/or client device 400. Generally, an integration engine 130 may enable or facilitate the transfer of data between the one or more user interface engines 111 and records engines 121 to allow communication of data between one or more EMR systems 110 and NMCC systems 120. In some embodiments, an integration engine 130 may be configured to: receive input from a user 101 via user interface engine 111 containing a query for social data of a patient via a EMR graphical user interface 114 generated by a user interface engine 111; use the input to query one or more non-medical care coordination systems 120 for social data of the patient; and then return the data for display to the user 101 in the EMR graphical user interface 114 (optionally by generating a NMCC graphical user interface 124 within the EMR graphical user interface 114). In further embodiments, an integration engine 130 may be configured to receive input from a user 101 via user interface engine 111 containing data for the creation of a social referral 127 for a patient via a EMR graphical user interface 114 generated by a user interface engine 111; use the input to create a social referral 127 in one or more non-medical care coordination systems 120 for the patient; and then return social referral 127 data for display to the user 101 in the EMR graphical user interface 114 (optionally by generating a NMCC graphical user interface 124 within the EMR graphical user interface 114). In still further embodiments, an integration engine 130 may be configured to receive input from a user 101 via user interface engine 111 containing data for creating, editing, or otherwise modifying a non-medical care record 123 for a patient in the NMCC database 122 via a EMR graphical user interface 114 generated by a user interface engine 111; use the input to create, edit, or otherwise modify the data of the non-medical care record 123 in one or more non-medical care coordination systems 120 for the patient; and then return the created, edited, or otherwise modified the data of the non-medical care record 123 data for display to the user 101 in the EMR graphical user interface 114 (optionally by generating a NMCC graphical user interface 124 within the EMR graphical user interface 114).

As an example, a user 101, such as a medical professional 101A, look up a patient medical record 113 of a patient on an EMR system 110, via EMR graphical user interface 114 generated by a user interface engine 111, and determines that this patient might have a social need. The user 101 may then access the NMCC system 120, such as by selecting a social icon 125, which may be displayed on the EMR graphical user interface 114.

The integration engine 130 may then use information in the patient medical record 113, such as an ID number of the patient, to query the non-medical care coordination system 120 for information associated with the patient in the NMCC database 122. The records engine 121 may look up the patient in the NMCC database 122, such as by using the patient ID number. If the patient is found in the NMCC database 122, the records engine 121 may access that non-medical care records 123 within the NMCC database 122 and retrieves the patient's social data. This data may then be provided to the user interface engine 111 via the integration engine 130, and the social data may be displayed as a social data frame 126 within the EMR graphical user interface 114.

If the patient is not found in the NMCC database 122, the records engine 121 may use other data from the patient medical record 113 of the EMR database 112 and may search for matching patient(s) or individual(s) within the NMCC database 122 based on that data. Any matches found may be presented to the user 101 via the EMR graphical user interface 114, such as in a social data frame 126 and the user 101 may then select which patient to use.

If no match is found or in case the user 101 does not want to use any of the matches found, the user 101 may provide data which may be used to generate a new patient non-medical care record 123 in the NMCC database 122. To save time, preferably this non-medical care record 123 may include all or some of the patient data retrieved from the EMR system 110.

Once the non-medical care record 123 of the patient has been identified, the user 101 may start a care episode which typically starts by creating a new social referral 127 for the patient for a specific service.

In some embodiments, the user 101 might want to start by analyzing the patient's needs and identify the patient's needs first and then create social referrals 127 to services that address those needs. In further embodiments, analyzing the patient's needs might be done based on a questionnaire that may be completed by an individual, such as the patient, care giver, or medical professional 101A. Analyzing the patient's needs might also take into account the patient's data of their patient medical record 113 retrieved from the EMR system 110 and previous medical and non-medical care episodes, previous questionnaires filled in for the same patient

In some embodiments, the questionnaire questions may be stored in a database, such as an EMR database 112 and/or NMCC database 122. In further embodiments, one or more questionnaire questions may be configured to be dynamic so that certain questions might be displayed based on the answers provided to earlier questions. When the same patient answers the same questionnaire several times, an analysis that shows that patient's progress over time might be presented by an engine 111, 121, 130.

In some embodiments, an EMR system 110 may comprise its own questionnaires that result with identified need(s), and the system 100 may be configured to pass the list of identified needs to the non-medical care coordination system 120 that can create a social referral 127 for services that answer the identified need(s) during or after a questionnaire of the EMR system 110 is being filled in. Optionally, when a need is identified or in case the user 101 knows what the need is, the user 101 will be able to create a social referral 127 for the patient in the system 100. As the patient data is already available, via the patient medical record 113, this data can be reused for the social referral 127.

In some embodiments, any changes made to the social referral 127, as well as any additional data elements that are derived of the referral data, may be reflected on a user interface 114, 124, such as an EMR graphical user interface 114 of the EMR system 110. This may be achieved by modifying the data that is stored by the EMR system 110 in an EMR database 112 and/or can be achieved by showing that referral data as well as derived data through the social data frame 126 that may be used to show the NMCC graphical user interface 124 and the associated social data of a NMCC database 122.

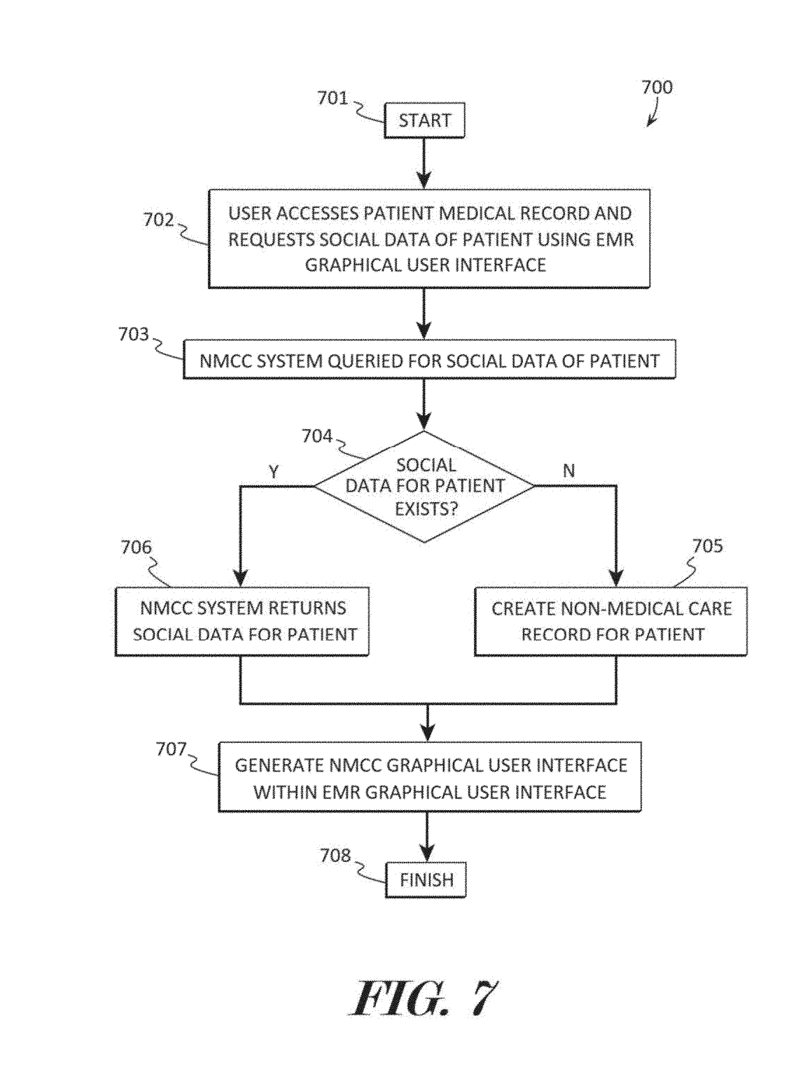

FIG. 7 shows a block diagram of an example of a computer-implemented method for accessing social data of a patient (“the method”) 700 according to various embodiments described herein. In some embodiments, the method 700 may be used to receive and provide social data of a patient via a NMCC graphical user interface 124 which may be embedded within an EMR graphical user interface 114 of the system 100. One or more steps of the method 700 may be performed by a user interface engine 111, records engine 121, and/or integration engine 130 which may be executed by a computing device processor, such as a processor 302 (FIG. 2) and/or a processor 402 (FIG. 3). The functions attributed to an engine 111, 121, 130, are not exclusive to that engine 111, 121, 130, and in other embodiments, one or more functions attributed to an engine 111, 121, 130, may be performed by one or more other engines 111, 121, 130.

In some embodiments, the method 700 may start 701 and a user 101, such as a medical professional 101A, may access a patient medical record 113 and may request social data of the patient using an EMR graphical user interface 114 of an EMR system 110 in step 702. In some embodiments, an integration engine 130 may send the request from the EMR system 110 to a NMCC system 120, the request to present social data of the patient via the EMR graphical user interface 114 of that EMR system 110. In further embodiments, the EMR system 110 may launch a NMCC system 120 which then retrieves and displays the data on the EMR graphical user interface 114. In preferred embodiments, the EMR graphical user interface 114 may be generated on a display screen 404A of a client device 400 by a user interface engine 111. The user 101 may look up the patient medical record 113 of the patient on an EMR system 110, and may determine that this patient may have a social need. In further embodiments, a social icon 125 may be generated by the user interface engine 111 on the EMR graphical user interface 114, and the user 101 may select the social icon 125 to request social data of the patient.

In step 703, the NMCC system 120 may query for the social data of patient. In preferred embodiments, the data of the patient or the patient context that may be contained in the patient medical record 113 may be used in the query provided to the NMCC system 120. For example, the patient medical record 113 may contain a patient identification (ID) number, and the query provided to the NMCC system 120 may contain the ID number which the NMCC system 120 may use to search a NMCC database 122.

In decision block 704, the system 100 may determine if social data of the patient exists in the NMCC database 122. Preferably, the records engine 121 may search the non-medical care records 123 of the NMCC database 122 for data matching the patient. If a matching non-medical care record 123 is found, the system 100 may determine that social data of the patient exists, and the method 700 may proceed to step 706. If a matching non-medical care record 123 is not found, the system 100 may determine that social data of the patient does not exist in the NMCC system 120, and the records engine 121 may create a non-medical care record 123 for patient in the NMCC database 122 preferably using data from the patient medical record 113 in step 705. The method 700 may then proceed to step 707.

In step 706, the NMCC system 120 may return social data for patient that may be contained in a non-medical care record 123. In some embodiments, the records engine 121 may provide the social data to the interface engine 111 via the integration engine 130. If only one or more partially matching non-medical care records 123 for the patient are found in the NMCC database 122, any matching non-medical care records 123 may be returned in step 706.

In step 707, a NMCC graphical user interface 124 may be generated within an EMR graphical user interface 114 to allow the user 101 to view the social data of the patient, such as by viewing past social referrals 127 or any other data of the patient's social data, and add or edit the social data, such as by generating a social referral 127 for the patient. In preferred embodiments, the NMCC graphical user interface 124 may be embedded within a social data frame 126 of the EMR graphical user interface 114 so that the NMCC graphical user interface 124 and EMR graphical user interface 114 are simultaneously displayed on the display screen 404A of the client device 400. Additionally, the user 101 may view the social data for screening purposes. Optionally, one or more engines 111, 121, 130, may generate the NMCC graphical user interface 124 within a social data frame 126 of the EMR graphical user interface 114. In preferred embodiments, social data of a non-medical care record 123 may be displayed to the user via a NMCC graphical user interface 124 that may be generated within an EMR graphical user interface 114. In some embodiments, any partially matching non-medical care records 123 found in step 706 may be presented to the user 101 via the EMR graphical user interface 114, such as in a social data frame 126, and the user 101 may then select which patient to use. If no match is found or in case the user 101 does not want to use any of the matches found, the user 101 may provide data which may be used to generate a new patient non-medical care record 123 in the NMCC database 122. To save time, preferably this non-medical care record 123 may include all or some of the patient data retrieved from the EMR system 110. After step 707, the method 700 may finish 708.

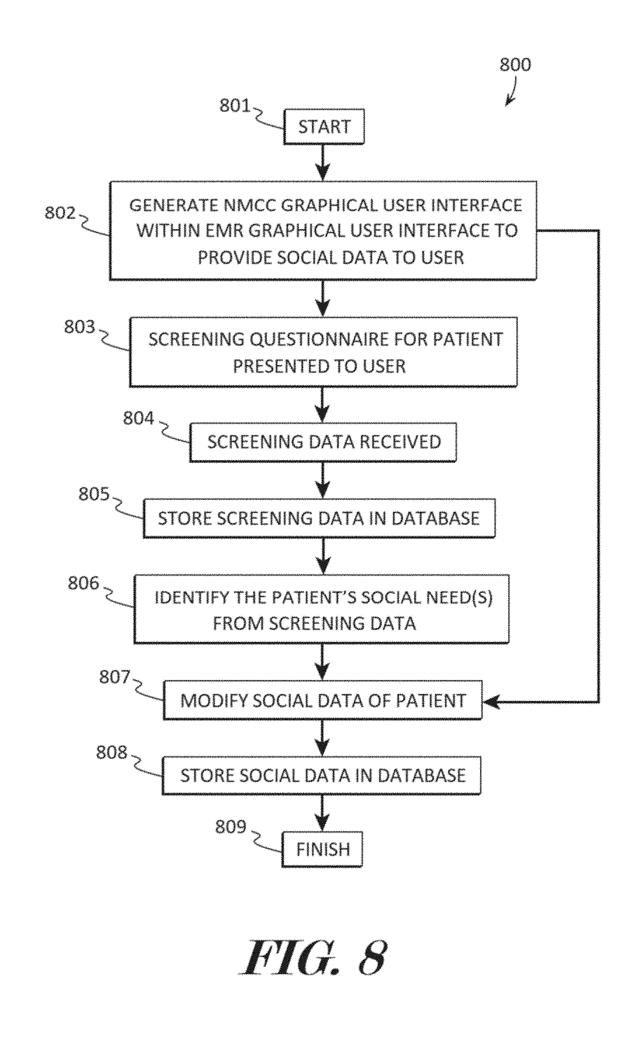

FIG. 8 shows a block diagram of an example of a computer-implemented method for modifying social data of a patient (“the method”) 800 according to various embodiments described herein. In some embodiments, the method 800 may be used to created, edit, or otherwise modify social data of a patient via a NMCC graphical user interface 124 which may be embedded within an EMR graphical user interface 114 of the system 100. One or more steps of the method 800 may be performed by a user interface engine 111, records engine 121, and/or integration engine 130 which may be executed by a computing device processor, such as a processor 302 (FIG. 2) and/or a processor 402 (FIG. 3). The functions attributed to an engine 111, 121, 130, are not exclusive to that engine 111, 121, 130, and in other embodiments, one or more functions attributed to an engine 111, 121, 130, may be performed by one or more other engines 111, 121, 130.

In some embodiments, the method 800 may start 801 and a NMCC graphical user interface 124 may be generated within an EMR graphical user interface 114 on a display screen 404A of a client device 400 to provide social data of a patient to user 101, such as a medical professional 101A in step 802. In preferred embodiments, a NMCC graphical user interface 124 may be generated within an EMR graphical user interface 114 according to the method of accessing social data of a patient 700 of FIG. 7. In preferred embodiments, the NMCC graphical user interface 124 may be embedded within a social data frame 126 of the EMR graphical user interface 114 so that the NMCC graphical user interface 124 and EMR graphical user interface 114 are simultaneously displayed on the display screen 404A of the client device 400.

In optional step 803, a screening questionnaire for the patient may be presented to the user 101. In some embodiments, a user 101 may interact with a user interface 114, 124, and an engine 111, 121, 130, such as an interface engine 130, may retrieve a questionnaire from a database 112, 122. To be presented to the user 101. Optionally, a questionnaire may be presented to the user 101 and/or a patient via a user interface 114, 124, on a display screen 404A of a client device 400 and/or a physical copy of a questionnaire may be presented to the user 101 and/or a patient via a printer or other printing device. In further embodiments, one or more questionnaire questions may be configured to be dynamic so that certain questions might be displayed based on the answers provided to earlier questions. When the same patient answers the same questionnaire several times, an analysis that shows that patient's progress over time might be presented by an engine 111, 121, 130.

In optional step 804, screening data from the screening questionnaire may be received. In some embodiments, the questionnaire may be completed by an individual, such as the patient, care giver, or medical professional 101A, and the screening data may be received by an engine 111, 121, 130, such as an interface engine 130, optionally via a user interface 114, 124, and/or upon being converted or otherwise received in a digital format.

In optional step 805, the screening data may be stored in a database 112, 122. In some embodiments, an interface engine 130 may provide the screening data to a user interface engine 111, and the user interface engine 111 may store the screening data in an EMR database 112. In further embodiments, an interface engine 130 may provide the screening data to a records engine 121, and the user interface engine 111 may store the screening data in a NMCC database 122. In further embodiments the screening data from the questionnaire responses may be communicated to the NMCC system 120 by the user interface engine 111 of the EMR system 110.

In optional step 806, the patient's social need(s) may be identified from the screening data. In some embodiments, the patient's social need(s) may be identified by the user 101 and/or an engine 111, 121, 130, such as an interface engine 130. In preferred embodiments, an interface engine 130 may analyze the screening data to identify the patient's needs and optionally may also take into account the patient's data of their patient medical record 113 retrieved from the EMR system 110. In further embodiments the identified social need(s) may be communicated to the NMCC system 120 by the user interface engine 111 of the EMR system 110.

In step 807, the social data of the patient may be modified. In some embodiments, the social data of the patient may be modified by one or more social referrals 127 for the patient being created, edited, or otherwise modified, preferably for one or more need(s), such as which may be identified in optional step 805. In further embodiments, the social data of the patient may be modified by one or more social conditions, social needs, care episodes, social assessments, cases, intakes, assistant requests, or any other social data for the patient being created, edited, or otherwise modified. In some embodiments, the user 101 may interact with a graphical user interface 114, 124, to cause an engine 111, 121, 130, such as a records engine 121, to generate one or more social referrals 127. Preferably, the user 101 may start a care episode which typically starts by creating a new social referral 127 for the patient for a specific service. In further embodiments, an engine 111, 121, 130, such as a records engine 121, may generate one or more social referrals 127. In still further embodiments, any other social data of the patient that may be stored in a non-medical care record 123 may be modified.

In step 807, the social data for the patient may be stored in a database, such as in a non-medical care record 123 of the patient in a NMCC database 122 and/or a patient medical record 113 of the patient in an EMR database 112. In preferred embodiments, the social data may include one or more social referral(s) 127 which may have been created in step 807. After step 808, the method 800 may finish 809.

In some embodiments of method 800, the screening questionnaire may be stored in and retrieved from the NMCC database 122 in step 803, the responses may be captured through the NMCC graphical user interface 124 that may be embedded in the EMR graphical user interface 114 in step 804, and the screening data may be analyzed to identify needs in step 806 by the NMCC system 120.

In some embodiments of method 800, the screening questionnaire may be stored in and retrieved from the EMR database 112 in step 803, the responses may be captured through the EMR graphical user interface 114 and sent to the NMCC system 120 in step 804, and the screening data may be analyzed to identify needs in step 806 by the NMCC system 120.

In some embodiments of method 800, the screening questionnaire may be stored in and retrieved from the EMR database 112 in step 803, the responses may be captured through the EMR graphical user interface 114 in step 804, and the screening data may be analyzed to identify needs in step 806 by the EMR system 110, and the identified needs may be sent to the NMCC system 120.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine-readable storage device, a machine-readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), light emitting diode (LED) display, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random-access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD), light emitting diode (LED) display, or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g. through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and Wi-Fi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A computer-implemented method for accessing social data of a patient, the method comprising:
sending a request to a non-medical care coordination system (NMCC system), to present social data of a patient via an electronic medical records graphical user interface (EMR graphical user interface) of an electronic medical records system (EMR system), the EMR graphical user interface displayed on a display screen of a client device;
querying for social data of the patient via a non-medical care coordination system (NMCC system);
returning social data for the patient via from the NMCC system; and
generating a non-medical care coordination graphical user interface (NMCC graphical user interface) within the EMR graphical user interface, the NMCC graphical user interface displaying the social data on the display screen of the client device.

2. The method of claim 1, wherein the NMCC graphical user interface is embedded within a social data frame of the EMR graphical user interface.

3. The method of claim 1, wherein the NMCC graphical user interface is embedded within a social data frame of the EMR graphical user interface so that the NMCC graphical user interface and EMR graphical user interface are simultaneously displayed on the display screen of the client device.

4. The method of claim 1, wherein data from a patient medical record of the EMR system is used to query the NMCC system for social data of the patient.

5. The method of claim 1, wherein the request for social data of a patient is received by the user interacting with a social icon.

6. The method of claim 1, further comprising the step of creating a non-medical care record for the patient in the NMCC system using data from a patient medical record of the patient.

7. A computer-implemented method for modifying social data of a patient, the method comprising:
generating a non-medical care coordination graphical user interface (NMCC graphical user interface) of a non-medical care coordination system (NMCC system) within an electronic medical records graphical user interface (EMR graphical user interface) of an electronic medical records system (EMR system) to provide social data of a patient to a user via a display screen of a client device;
creating a social referral for the patient via the NMCC graphical user interface in the NMCC system.

8. The method of claim 7, wherein the NMCC graphical user interface is embedded within a social data frame of the EMR graphical user interface so that the NMCC graphical user interface and EMR graphical user interface are simultaneously displayed on the display screen of the client device.

9. The method of claim 7, further comprising the step of displaying a screening questionnaire for the patient to the user via the display screen of a client device.

10. The method of claim 7, further comprising the step of receiving screening data for the patient.

11. The method of claim 7, further comprising the step of identifying a social need for the patient from the screening data via an interface engine.

12. A computer-implemented patient care integration method, the method comprising:
sending a request to a non-medical care coordination system (NMCC system), to present social data of a patient via an electronic medical records graphical user interface (EMR graphical user interface) of an electronic medical records system (EMR system), the EMR graphical user interface displayed on a display screen of a client device;
querying for social data of the patient via a non-medical care coordination system (NMCC system);
returning social data for the patient via the NMCC system;
generating a non-medical care coordination graphical user interface (NMCC graphical user interface) within the EMR graphical user interface, the NMCC graphical user interface displaying the social data on the display screen of the client device; and
creating a social referral for the patient via the NMCC graphical user interface in the NMCC system.

13. The method of claim 12, wherein the NMCC graphical user interface is embedded within a social data frame of the EMR graphical user interface.

14. The method of claim 13, wherein the NMCC graphical user interface is embedded within a social data frame of the EMR graphical user interface so that the NMCC graphical user interface and EMR graphical user interface are simultaneously displayed on the display screen of the client device.

15. The method of claim 12, wherein data from a patient medical record of the EMR system is used to query the NMCC system for social data of the patient.

16. The method of claim 12, wherein the request for social data of a patient is received by the user interacting with a social icon.

17. The method of claim 12, further comprising the step of creating a non-medical care record for the patient in the NMCC system using data from a patient medical record of the patient.

18. The method of claim 12, further comprising the step of displaying a screening questionnaire for the patient to the user via the display screen of a client device.

19. The method of claim 12, further comprising the step of receiving screening data for the patient.

20. The method of claim 12, further comprising the step of identifying a social need for the patient from the screening data via an interface engine.
